SEQUENCE LISTING (28 sequences)

SEQ ID NO: 1 - DNA, 20 bp, synthetic construct
  source 1..20: mol_type = other DNA
cgaggcagtatcactcattg

SEQ ID NO: 2 - DNA, 20 bp, synthetic construct
  source 1..20: mol_type = other DNA
cgttgcttggttctccttgt

SEQ ID NO: 3 - DNA, 20 bp, synthetic construct
  source 1..20: mol_type = other DNA
aactttggcattgtggaagg

SEQ ID NO: 4 - DNA, 20 bp, synthetic construct
  source 1..20: mol_type = other DNA
cacattgggggtaggaacac

SEQ ID NO: 5 - DNA, 20 bp, synthetic construct
  source 1..20: mol_type = other DNA
agacccctgtgctccaagtg

SEQ ID NO: 6 - DNA, 20 bp, synthetic construct
  source 1..20: mol_type = other DNA
cagactccatttgccagcag

SEQ ID NO: 7 - DNA, 18 bp, synthetic construct
  source 1..18: mol_type = other DNA
gaaccgccccttatcaag

SEQ ID NO: 8 - DNA, 21 bp, synthetic construct
  source 1..21: mol_type = other DNA
caggatgtccctgctctcctt

SEQ ID NO: 9 - DNA, 23 bp, synthetic construct
  source 1..23: mol_type = other DNA
tgatcccaatgagtaggctggag

SEQ ID NO: 10 - DNA, 23 bp, synthetic construct
  source 1..23: mol_type = other DNA
atgtctggacccattccttcttg

SEQ ID NO: 11 - DNA, 20 bp, synthetic construct
  source 1..20: mol_type = other DNA
tcccttctcaccaccagtta

SEQ ID NO: 12 - DNA, 20 bp, synthetic construct
  source 1..20: mol_type = other DNA
cagtcttgtgggcactctaa

SEQ ID NO: 13 - DNA, 20 bp, synthetic construct
  source 1..20: mol_type = other DNA
aatcgacaacaacccctttg

SEQ ID NO: 14 - DNA, 20 bp, synthetic construct
  source 1..20: mol_type = other DNA
aactgtgttcccgaggtgtc

SEQ ID NO: 15 - DNA, 23 bp, synthetic construct
  source 1..23: mol_type = other DNA
atttgaattccctgggtgagaag

SEQ ID NO: 16 - DNA, 21 bp, synthetic construct
  source 1..21: mol_type = other DNA
cacaggggagaaatcgatgac

SEQ ID NO: 17 - DNA, 23 bp, synthetic construct
  source 1..23: mol_type = other DNA
aggataccactcccaacagacct

SEQ ID NO: 18 - DNA, 24 bp, synthetic construct
  source 1..24: mol_type = other DNA
caagtgcatcatcgttgttcatac

SEQ ID NO: 19 - DNA, 20 bp, synthetic construct
  source 1..20: mol_type = other DNA
caatgaacgctacacactgc

SEQ ID NO: 20 - DNA, 20 bp, synthetic construct
  source 1..20: mol_type = other DNA
ccacatctatgccacttgag

SEQ ID NO: 21 - DNA, 21 bp, synthetic construct
  source 1..21: mol_type = other DNA
ccccaaagggatgagaagttc

SEQ ID NO: 22 - DNA, 20 bp, synthetic construct
  source 1..20: mol_type = other DNA
tgagggtctgggccatagaa

SEQ ID NO: 23 - DNA, 20 bp, synthetic construct
  source 1..20: mol_type = other DNA
tcaggcaggcagtatcactc

SEQ ID NO: 24 - DNA, 19 bp, synthetic construct
  source 1..19: mol_type = other DNA
ctaatgggaacgtcacacc

SEQ ID NO: 25 - DNA, 25 bp, synthetic construct
  source 1..25: mol_type = other DNA
cagaagctaaccatctcctggtttg

SEQ ID NO: 26 - DNA, 25 bp, synthetic construct
  source 1..25: mol_type = other DNA
tccggagtaatttggtgcttcacac

SEQ ID NO: 27 - DNA, 19 bp, synthetic construct
  source 1..19: mol_type = other DNA
gtgycagcmgccgcggtaa

SEQ ID NO: 28 - DNA, 20 bp, synthetic construct
  source 1..20: mol_type = other DNA
ccgycaattymtttragttt

(19) United States
(12) Patent Application Publication
(10) Pub. No.: US 2025/0360177 A1
(43) Pub. Date: Nov. 27, 2025

(54) FERMENTATION PRODUCT FOR THE TREATMENT OF INFLAMMATORY DISEASES

(71) Applicant: ALFASIGMA S.P.A., Bologna (IT)

(72) Inventors: Loredana Vesci, Pomezia (RM) (IT); Fabrizio Giorgi, Pomezia (RM) (IT)

(73) Assignee: ALFASIGMA S.P.A., Bologna (IT)

(21) Appl. No.: 18/875,012

(22) PCT Filed: Jun. 30, 2023
(86) PCT No.: PCT/EP2023/068130

§ 371 Date: Dec. 13, 2024

(30) Foreign Application Priority Data
Jul. 4, 2022 (IT) 102022000013882

(51) Int. Cl. A61K 36/48; A61K 35/747; A61K 36/31; A61K 36/324; A61K 36/539; A61K 36/82; A61K 36/9066; A61P 1/00; A61P 29/00

(52) U.S. Cl. CPC A61K 36/48; A61K 35/747; A61K 36/31; A61K 36/324; A61K 36/539; A61K 36/82; A61K 36/9066; A61P 1/00; A61P 29/00; A61K 2236/19; A61K 2236/51; A61K 2236/53

(57) ABSTRACT

The present invention relates to a fermentation product obtained by a fermentation process of at least one bacterial strain in the presence of at least one vegetable extract. The fermentation product of a bacterial may comprise alimentary and/or pharmaceutically acceptable components or comprised in medical food or nutritional composition. The fermentation product and is useful for the treatment and/or prevention of inflammatory diseases, in particular, inflammatory bowel disease.

### FIELD OF THE INVENTION

The present invention relates to a fermentation product obtained by a fermentation process of at least one bacterial strain in the presence of at least one vegetable extract. The fermentation product of a bacterial may comprise alimentary and/or pharmaceutically acceptable components or comprised in medical food or nutritional composition. The fermentation product and is useful for the treatment and/or prevention of inflammatory diseases, in particular inflammatory bowel disease.

### STATE OF THE ART

The gut microbiota consists of the set of microorganisms present in the human gut. It consists of more than one thousand pathogenic or symbiotic bacterial species, of which one hundred and fifty to five hundred reside in the colon. The microbiota establishes a commensal and mutualistic relationship with the host by digesting carbohydrates, oligosaccharides, and producing certain types of vitamins, such as B group vitamins, K group vitamins, and folates. In addition, the gut microbiota interacts with the innate and adaptive immune system through the production of bacteria-associated molecular patterns (MAMPs) recognized by specific gut immune cell receptors (Becattini S. et al., Trends Mol Med, 2016 22:458-78).

The alteration in the quantity and quality of bacterial species compared to the symbiotic condition of the microbiota of a healthy subject is called dysbiosis. Specifically, bacterial dysbiosis can inhibit the expression of epithelial junction proteins and impair bacterial recognition by the immune system thus leading to an aberrant immune response.

Several therapeutic strategies have been developed to counter gastrointestinal disorders by modulating the microbiota, such as the use of prebiotics (foods that cannot be digested by humans but are beneficial to the microbiota), probiotics (living microorganisms administered orally), postbiotics (metabolic products derived from probiotics), or fecal microbiota transplants from a healthy donor.

In particular, probiotics are commonly used as adjuvants in the treatment of inflammatory bowel disease, allergic phenomena, urinary tract infections, colon cancer, hypercholesterolemia, and constipation. Lactobacilli are bacteria naturally present in the human body that can be taken in the form of food (particularly yogurt and other fermented dairy products) or dietary supplements, and they perform several useful functions for human health. They can, for example, contribute to food digestion, nutrient absorption, and act against pathogenic microorganisms that can trigger various problems in humans and animals. Their intake is proposed for use in intestinal disorders and to prevent them during antibiotic therapy, in the treatment and/or prevention of diarrhoea, irritable bowel syndrome, colic in children, Crohn's disease, and necrotizing enterocolitis. In addition, they are proposed for use in the treatment of gastric Helicobacter pylori infections, urinary tract infections, and vaginal infections. Finally, lactobacilli are sometimes taken to prevent cold diseases, to reduce high cholesterol levels, in Lyme disease, and dermatological diseases.

Many studies report that probiotics may play important roles in maintaining intestinal homeostasis by modulating immunity and increasing epithelial barrier function. Clinical studies and systemic meta-analyses have shown that certain strains of probiotics have beneficial effects in selected patients. Beom J. L. et al. describes in J Neurogastroenterol Motil. 2011, 17(3), 252-266, that the use of probiotics has long been an alternative to conventional medicine for the treatment of many diseases, such as in the treatment of IBS. In the context of dysbiosis as a pathogenesis of IBS, probiotic treatment seems reasonable and perhaps ideal as it restores the gut microbiota.

Guendalini S in Front. Med., Aug. 28, 2014 describes the use of Lactobacillus GG, Lactobacillus reuteri DSM 17938 and probiotic mixture VSL #3 in pediatric patients with predominant diarrhea or post-infectious IBS.

Liu Yang in Engineering 2021, Volume 7, Issue 3, 376-38 describes that the use of Lactobacillus plantarum CCFM8610 can significantly alleviate clinical symptoms and dysbiosis of the gut microbiota in patients with IBS-D. The alleviation effect of IBS-D given by L. plantarum CCFM8610 may be related to the increase in the relative abundance of butyric acid-producing genera in the intestine.

Park J S et al. in J Med Food 2018,21(3), 215-224 reports that treatment with L. acidophilus attenuated the severity of sodium dextran sulfate (DSS)-induced colitis. Specifically, L. acidophilus suppresses proinflammatory cytokines produced by T helper (Th) 17 cells in colonic tissues, such as interleukin-6 (IL-6), tumor necrosis factor-α, IL-1β and IL-17. In addition, in vitro treatment with L. acidophilus directly induces regulatory T cells (Treg) and IL-10 production, while IL-17 production is suppressed in splenocytes.

Hill C et al. in Nat. Rev. Gastroenter. Hepatol 2014, 11 (8), 506 describes that administration of live bacteria confers a benefit to the host. Probiotics confer beneficial actions through a variety of mechanisms by acting on immunomodulation, pain, and changing nervous system signals. Specifically, probiotics are able to integrate into the gut microbiota and influence its composition and activity, including stimulation of the existing microbiota through trophic interactions, inhibition and/or reduction of pathogens by changes in the gut microbial environment.

Phytotherapeutic treatments, particularly in Chinese medicine, are also useful in preventing and treating disorders related to intestinal inflammation. Herbal therapies exert their therapeutic benefit through several mechanisms including immune regulation, antioxidant activity, inhibition of leukotriene B4 and nuclear factor-kappa B (NF-KB), and antiplatelet activity.

Triantafyllidi A et al. in Ann Gastroenterol. 2015, 28(2):210-220 reports that the most important clinical trials conducted to date relate to the use of mastic gum, tormentil extracts, wormwood herb, aloe vera, Triticum aestivum, germinated barley, and Boswellia serrata. In ulcerative colitis, aloe vera gel, Triticum aestivum, Andrographis paniculata extract, and topical Xilei-san are superior to placebo in inducing remission or clinical response, and curcumin is superior to placebo in maintaining remission; Boswellia serrata in the form of gommoresin and Plantago ovata seeds are as effective as mesalazine, while Oenothera biennis has similar relapse rates as ω-3 fatty acids in treating ulcerative colitis. In Crohn's disease, mastic gum, Artemisia absinthium and Tripterygium wilfordii are superior to placebo in inducing remission and preventing postoperative clinical recurrence, respectively.

Lopes Andrade A. W. et al. in Front. Pharmacol., Jul. 29, 2020, 998 reports the beneficial effect of herbal products rich in bioactive compounds with immunomodulatory and antioxidant properties, as in the case of Bryophyllum pinnatum (Crassulaceae). This plant is used in traditional Brazilian medicine to treat inflammatory diseases.

Flavonoid compounds are hydroxylated polyphenolic molecules abundant in plants, including vegetables and fruits that are the main food sources of these compounds for humans, along with wine and tea. Flavonoids are of great interest because of their beneficial effects on health and in disease prevention. Most interest is directed toward their antioxidant activity, highlighting a remarkable free radical scavenging ability. However, accumulating evidence suggests that flavonoids have many other biological properties, including anti-inflammatory, antiviral, anticancer, and neuroprotective activities through different mechanisms of action.

In addition to plants, some foods traditionally consumed in the East, such as legumes, for example, green bean or mung bean (Phaseolus radiatus or Vigna radiata L.), are used to treat or prevent some diseases. Kalim A et al. in Journal of Pharmacognosy and Phytochemistry 2021; 10(2), 54 reports that Vigna radiata is one of the most important legume crops, grown from tropical to subtropical areas around the world. It is reported that green bean helps in the prevention of the risk of hypercholesterolemia, coronary heart disease and decreases the absorption of toxic substances.

Tang et al. Chemistry Central Journal 2014, 8:4 describes the antioxidant, antimicrobial, anti-inflammatory, antidiabetic, antihypertensive, lipid metabolism accommodation, antihypertensive and anticancer effects of Vigna radiata commonly used for its medicinal activities. Green bean sprouts contain a higher concentration of nutrients and bioactive compounds than the seed. The sprouts contain polyphenols, polysaccharides, polypeptides, and isoflavones, which possess pharmacological properties, such as hypoglycemic, hypolipidemic, antihypertensive, anticancer, hepatoprotective, and immunomodulatory activities, assayed both in vitro and in animal models.

Toledo O. et al. in Food Chemistry 127(3), 1175-1185 reports that hydrolysates of the common bean can be used to combat inflammatory and oxidation-associated diseases by demonstrating that the bean significantly inhibits NF-κB trans-activation and nuclear translocation of the NF-κB p65 subunit.

Chao WW et al. in Journal of Medicinal FoodVol. 18, No. 7 reports that red bean exerts an anti-inflammatory response and has potential as a beneficial ingredient and describes that at concentrations of 50-200 μg/mL it can significantly suppress inflammatory responses in LPS-stimulated macrophages through reduction of cellular NO and downregulation of gene expressions of iNOS, COX-2, TNF-α and IL-6 in a dose-dependent manner. In addition, it can decrease H₂O₂-induced oxidative damage in macrophage RAW 264.7.

The activity of plants or plant extracts can be affected by the presence of bacteria. C G Rizzello et al. in Microbial Cell Factories 2013, 12:44 reports that plant extracts fermented with lactobacilli have beneficial effects on the organism. For example, fermentation of Echinacea and L. plantarum is reported to have better antimicrobial effects than Echinacea alone.

Oh N. S. et al., J. Dairy Sci., 2020, 103:2947-2955 reports that the use of milk fermented with extracts of C. tricuspidata and Lactobacillus gasseri 505 has greater antioxidant properties than fermentation of C. tricuspidata alone, and the resulting product has hepatoprotective effects on colorectal cancer and in the induction of liver metastasis.

WO 2020/0070087 describes the combination of a Bifidobacterium longum with a Bifidobacterium lactis for use in the treatment of Crohn's disease in which the probiotic composition increases the concentration of anti-inflammatory cytokines.

US 2007/298019 describes a composition for the treatment of inflammatory bowel disease containing Lactobacillus delbrueckii ssp. Bulgaricus and plant components.

KR 2021-0128947 describes the use of a Lactobacillus sakei strain capable of converting soybean roots or extracts into the isoflavone metabolites useful for their antioxidant properties.

However, remains a need to have alternative compositions, well accepted by patients, characterized by anti-inflammatory activity, particularly with intestinal anti-inflammatory activity for human and/or animal use.

There is also a need for effective compositions especially of natural origin that are useful to be administered as an alternative and/or in combination with antibiotics, to reduce their consumption and not create antibiotic resistance, and that are well tolerated by patients and do not create resistance. Nutraceutical compositions are also useful for administration in patients undergoing other therapies, where additional antibiotic therapy could have adverse effects, as well as in immunocompromised patients and pediatric patients.

Surprisingly, has been found and is the subject of the present invention,.a nutraceutical composition comprising a fermentation product obtained by a fermentation process of at least one bacterial species in the presence of at least one plant extract The composition of the invention comprising the fermentation product combines the efficacy of Lactobacillus and plant matrix metabolites obtained for its action.

The composition of the invention is useful for the treatment and/or prevention of inflammatory diseases, such as inflammatory bowel disease.

### SUMMARY OF THE INVENTION

It is an object of the invention a fermentation product obtained by a fermentation process of Lactobacillus brevis strain, deposited with the Leibniz Institute DSMZ-German Collection of Microorganisms and Cell Cultures GmbH, identified by DSM number 33682, in the presence of at least one plant extract chosen from the group consisting of: Scutellaria baicalensis Georgi, Boswellia serrata, Phaseolus, Curcuma longa, Camellia sinensis, broccoli sprouts and mixtures thereof.

It is an object of the invention to have a composition comprising a fermentation product obtained by the fermentation of the bacterial strain Lactobacillus brevis deposited at the Leibniz Institute DSMZ-German Collection of Microorganisms and Cell Cultures center, registered under accession number DSM 33682 in the presence of at least one plant extract chosen from the group consisting of: Scutellaria baicalensis Georgi, Boswellia serrata, Phaseolus, Curcuma longa, Camellia sinensis, broccoli sprouts and mixtures thereof.

It is an object of the invention to provide a process for obtaining the fermentation product of the Lactobacillus brevis strain, deposited at the Leibniz Institute DSMZ-German Collection of Microorganisms and Cell Cultures GmbH, identified by DSM number 33682, in the presence of at least one plant extract chosen from the group consisting of: Scutellaria baicalensis Georgi, Boswellia serrata, Phaseolus, Curcuma longa, Camellia sinensis, broccoli sprouts and mixtures thereof.

It is an object of the invention to provide a process for obtaining a pharmaceutical or nutraceutical composition comprising the fermentation product according to the present invention obtained in the presence of at least one plant extract together with pharmaceutical or food excipients.

### BRIEF DESCRIPTION OF THE FIGURES

FIG. 1: Colon Damage score and Disease activity index of mice treated with Prob1. T-test for Vehicles vs TNBS was first applied with a p-value <0.001 (⋅⋅⋅) and p<0.0001 (⋅⋅⋅⋅). A One Way Anova (or equivalent) was applied to com-pare all other groups to TNBS with a p-value <0.05 (*), p<0.01 (**), p<0.001 (***) and p<0.0001 (****).

FIG. 2: MPO levels measured in colon of TNBS-induced colitis-treated mice. T-test for Vehicles vs TNBS was first applied with a p-value <0.0001 (⋅⋅⋅⋅). A One Way Anova (or equivalent) was applied to compare all other groups to TNBS with a p-value <0.01 (**), p<0.001 (***) and p<0.0001 (****).

FIG. 3: mRNA of pro-inflammatory cytokines measured in colon of TNBS-induced colitis treated mice. T-test for Vehicles vs TNBS was first applied with a p-value <0.01 (^{⋅⋅}). A One Way Anova (or equivalent) was applied to compare all other groups to TNBS with a p-value <0.05 (*), p<0.01 (**) and p<0.0001 (****).

FIG. 4: mRNA of immune cells measured in colon of TNBS-induced colitis treated mice. T-test for Vehicles vs TNBS was first applied with a p-value <0.01 (⋅⋅). A One Way Anova (or equivalent) was applied to compare all other groups to TNBS with a p-value <0.05 (*), p<0.01 (**) and p<0.0001 (****).

FIG. 5: Zonula occludens 1 protein levels measured in colon of TNBS-induced colitis treated mice. T-test for Ve-hicles vs TNBS was first applied with a p-value <0.0001 (⋅⋅⋅⋅). A One Way Anova (or equivalent) was applied to compare all other groups to TNBS with a p-value <0.05 (*) and p<0.01 (**).

FIG. 6: Protein levels of immune cells measured in colon of TNBS-induced colitis treated mice. T-test for Vehicles vs TNBS was first applied with a p-value <0.001 (⋅⋅⋅) and p<0.0001 (⋅⋅⋅⋅). A One Way Anova (or equivalent) was applied to compare all other groups to TNBS with a p-value <0.01 (**), p<0.001 (***) and p<0.0001 (****).

FIG. 7: Beta-diversity measured in stools of TNBS-induced colitis treated mice—(a) Dominant phyla; (b) Dominant families. T-test for Vehicles vs TNBS was first applied: p-value <0.05 (^{⋅⋅}). A One Way Anova (or equivalent) was applied to compare all other groups to TNBS with a p-value <0.05 (*) and p<0.01 (**).

### DETAILED DESCRIPTION OF THE INVENTION

The present invention describes a fermentation product obtained by a Lactobacillus strain fermentation process in the presence of at least one plant extract.

The present invention describes a fermentation product obtained by a fermentation process of the Lactobacillus brevis strain, deposited at the Leibniz Institute DSMZ-German Collection of Microorganisms and Cell Cultures GmbH, identified by DSM number 33682, in the presence of at least one plant extract selected from the group consisting of: Scutellaria baicalensis Georgi, Boswellia serrata, Phaseolus, Phaseolus radiatus, Phaseolus radiatus sprouts, Curcuma longa, Camellia sinensis, broccoli sprouts and mixtures thereof.

The product of fermentation can comprise probiotic Lactobacillus brevis DSM number 33682 in the presence of an amount of surnatant derived by the same fermentation process, in an amount of 0% to 30% (p/p) in comparison to the weight of the fermentation mass.

The amount of surnatant is regulated by the step of centrifugation, lyophilization or spray dray process.

The plant extracts useful for the invention are characterized by having antioxidant properties and can be in the form of a solid extract.

The probiotic Lactobacillus brevis DSM 33682 was placed in a culture medium, allowed to ferment, and the resulting product, characterized by 1×10⁸ to 1×10¹⁰ CFU/ml, was fermented in the presence of a plant matrix chosen from the group consisting of Scutellaria baicalensis Georgi, Boswellia serrata, Phaseolus radiatus sprouts, Curcuma longa, Camellia sinensis, broccoli sprouts and their mixtures.

The probiotic Lactobacillus brevis DSM 33682 was inoculated in an amount from 1×10⁸ to 1×10¹⁰ CFU/ml in a culture medium added with a plant matrix chosen from the group consisting of Scutellaria baicalensis Georgi, Boswellia serrata, Phaseolus, Phaseolus radiatus, Phaseolus radiatus sprouts, Curcuma longa, Camellia sinensis, broccoli sprouts and their mixtures.

One aspect of the present invention is the product obtained by fermentation of Lactobacillus brevis DSM 33682 in the presence of a Phaseolus extract. Preferably, the product is obtained by fermentation of Lactobacillus brevis DSM 33682 in the presence of a Phaseolus radiatus sprouts extract. More preferably, Phaseolus radiatus sprouts extract is in lyophilic form.

The product of the invention is obtained by inoculing L. brevis DSM 33682 in an amount from 1×10⁸ to 1×10¹⁰ CFU/ml in a culture medium added with an extract of a Phaseolus extract at a concentration from 5 to 15 grams/liter than the fermentation solution. Preferably, the product is obtained by fermentation of Lactobacillus brevis DSM 33682 in the presence of a Phaseolus radiatus sprouts extract at a concentration from 5 to 15 grams/liter than the fermentation solution.

The fermentation product according to the invention is characterised by being obtained by a process comprising the following steps:
(a) adding bacterial strain L. brevis DSM 33682 in culture medium in an amount of 1×10⁸ to 1×10¹⁰ CFU/ml;
b) adding the Phaseolus extract or Phaseolus radiatus extract or Phaesolus radiatus sprouds extract to the culture medium at a concentration of 5 to 15 grams/liter, based on the volume of the fermentation;
(c) keeping under mild agitation in anaerobic or aerobic conditions at temperatures between 30° C. and 40° C. for 8 to 24 hours;
(d) centrifuging at speeds lower than 4000 rpm for a time from 5 to 40 minutes;
(e) freeze-drying or freezing.

The culture medium can be any commercial medium suitable for the growth of Lactobacilli known to the expert in the art.

These media contain nutrients for bacterial growth, buffering agents, and are sterile to protect against any contamination. Soils may include:
(a) peptones: set of water-soluble compounds, obtained by hydrolysis (acid or enzymatic) of proteins (casein, soybean, etc.);
(b) NaCl: added in concentrations appropriate to the required osmotic needs;
(c) sugars: glucose, lactose, mannite, are added for specific purposes in particular soils;
(d) yeast, meat, organ extracts: provide growth factors and inorganic salts;
(e) enrichments: lysed blood, hemoglobin, dehydrated milk, gelatin vitamins.

Some media may include selective supplements, such as antibiotics, bile salts, and colorimetric indicators to follow the fermentative metabolism of the bacterium under investigation by colour-shifting of the medium at critical pH values.

The extract of Phaseolus or Phaseolus radiatus extract or Phaseolus radiatus sprout useful for preparing the fermentation product of the present invention is characterized by a redox potential from 100 to 400 mV when in phosphate buffer at concentrations from 0.5 to 20percent (w/v).

The fermentation product of L. brevis DSM 33682 in the presence of Phaseolus extract or Phaseolus radiatus extract or Phaseolus radiatus sprout extract can be obtained under anaerobic or aerobic conditions, preferably anaerobic, at a temperature from 30 to 40° C. for 8 to 24 h under low agitation. At the end of fermentation, the solution is centrifuged at speeds lower than 4000 rpm, preferably between 200 and 3000 rpm, for 5 to 40 minutes. Preferably, the solution is centrifuged at 3000 rpm for 10 minutes. The fermentation product resulting from the centrifugation of the fermentation solution is concentrated to a concentration factor from 6 to 10 times than the fermentation solution. The fermentation product can be frozen, freeze-dried or dried by spray drying.

The process for obtaining the fermentation product of L. brevis DSM 33682 in the presence of Phaseolus extract or Phaseolus radiatus extract or Phaseolus radiatus sprouts extract comprises the following steps:
(a) place L. brevis DSM 33682 in culture medium in an amount from 1×10⁸ to 1×10¹⁰ CFU/ml;
(b) add Phaseolus extract or Phaseolus radiatus extract or Phaseolus radiatus sprout extract to the culture medium at a concentration from 5 to 15 grams/liter than the fermentation solution;
(c) keep under low agitation under anaerobic or aerobic conditions at temperatures between 30° and 40° C. from 8 to 24 hours;
(d) centrifuge at speeds lower than 4000 rpm for a time from 5 to 40 minutes;
(e) freeze-dry or freeze.

The fermentation product of L. brevis DSM 33682 obtained in the presence of Phaseolus extract or Phaseolus radiatus extract or Phaseolus radiatus sprout extract under anaerobic conditions is characterized by having anti-inflammatory activity, determined in vitro by inhibition of IL-1beta mRNA transcription. It was observed that the fermentation product of L. brevis DSM 33682 obtained in the presence of Phaseolus radiatus sprout extract inhibited IL-1beta mRNA transcription at least three times more than the fermentation product obtained in the presence of the bacterium alone. Furthermore, the inhibition of IL-1beta mRNA transcription given by the fermentation product of L. brevis DSM 33682 obtained in the presence of Phaseolus radiatus sprout extract is comparable to the inhibition given by mesalazine.

The fermentation product of L. brevis DSM 33682 obtained in the presence of Phaseolus radiatus sprouts obtained under anaerobic or aerobic conditions, preferably anaerobic, is characterized by having anti-inflammatory activity in vivo. It can be in solid form obtained by lyophilization or spray drying.

It was surprisingly found that the fermentation product according to the invention in lyophilic form, when administered in an amount from about 1×10⁷ CFU/g to about 1×10⁹ CFU/g, restores colon length in an animal model with ulcerative colitis induced by 2,4,6, trinitrobenzenesulfonic acid (TNBS) to a greater extent than the group of animals treated with the fermentation product obtained in the absence of plant extracts and was found to be effective in a manner comparable to mesalazine.

The fermentation product in lyophilic form in an amount from about 10⁷ CFU to about 10⁹ CFU was administered to an animal model with ulcerative colitis induced by 2,4,6,trinitrobenzenesulfonic acid (TNBS) and was found to reduce the DAI score (disease activity index score used to assess colitis, including the parameters of reduced body weight and stool consistency), and counteracted the colon shortening (index of inflammation) of the animals in a manner comparable to that of the mesalazine-treated group of animals.

The fermentation product in lyophilic form according to the invention exerts a positive effect on TNBS-induced colonic injury in murine model. Importantly, the activity of Myeloperoxidase (MPO), a well-known marker of tissue injury and neutrophil infiltration able to induce mucosal disruption and ulceration with a close relationship with ulcerative colitis, decreased in the colon tissues of mice to a level comparable to the group treated with mesalazine

The expression levels of pro-inflammatory genes, including TNF-α and IL-1β, and levels of the anti-inflammatory gene as IL-10 in TNBS-induced colonic injury tissue in murine model was measured. IL-1β and TNF-α are believed to determine the degree of inflammation in IBD patients, in fact their increment positively correlate with the severity of IBD. The treatment of TNBS-induced acute colitis mice with mesalazine or the fermentation product of the invention reduced the expression levels of pro-inflammatory genes in mice.

It was also surprisingly found that the expression of IL-10, a key factor in the pathogenesis of IBD reducing the intestinal inflammation caused by TNBS, increased both in mesalazine and the fermentation product of the invention treatment groups in a comparable way.

Moreover, the fermentation product according to the invention significantly upregulates ZO-1 expression, a tight-junction protein, in the colons of TNBS-induced colitis mice to a level comparable to the group treated with mesalazine, following a reduction in their expression induced by TNBS.

The difference in gut microbiota of mice induced with TNBS was investigated. It was surprisingly found that the treatment with the fermentation product of the invention improved intestinal microbiota diversity in a way comparable to the group treated with mesalazine. In particular, at the phylum level, the administration of the product of the invention decreased the percentages of Proteobacteria, documented as responsible for the production of enterotoxins that often cause gastroenteritis or anaphylaxis, then the group treated with TNBS only.

At the family level, the relative abundance of Lachnospiraceae and Muribaculaceae, depleted by TNBS administration, was recovered and further enriched in mice treated with the fermentation product of the invention, with an improvement correlated to the ability to produce short-chain fatty acids and keeping eubiotic conditions, respectively recognized to these families. Significant differences were found in Lactobacilloae family, which were strongly reduced by the application of TNBS but significantly increased by the administration of the fermentation product of the invention. The capacity of Lactobacilliae to improve inflammatory bowel disease (IBD) and regulate the immune system is especially remarkable and well-known and due to several factors as production of protective molecules or downregulation in the production and release of pro-inflammatory cytokines (IL-6, IL-1β, IL-2 and TNF-α).

The activity of the fermentation product according to the present invention was compared in vivo versus the activity of other bacterial product, i.e. the probiotic mixture VSL #3 (consisting of Lactobacillus acidophilus, Lactobacillus plantarum, Lactobacillus casei, Lactobacillus delbrueckii subspecies bulgaricus, Bifidobacterium breve, Bifidobacterium longum, Bifidobacterium infantis, and Streptococcus salivarius subspecies thermophilus) used in keeping remission in patients with UC (Cheng et al., World J Clin Cases. 2020 Apr. 26; 8(8): 1361-1384) and E. coli Nissle used in preventing relapse in UC patients (Kruis et al., Gut. 2004 November; 53(11):1617-23). It was surprisingly found that the body weight loss of the mice treated with the fermentation product of the present invention was up to 50% lower than the mice treated with VSL #3 or E. coli Nissle. The activity was demonstrated also measuring the DAI score, colon damage score and the colon length, which improved in the mice treated with the product of the present invention in comparison to VSL #3 and E. coli Nissle treatment groups, in a dose dependent way.

Another object of the invention is a nutraceutical or food composition comprising the fermentation product of L. brevis (DSM 33682) obtained in the presence of a plant extract of Phaseolus or Phaseolus radiatus, preferably Phaseolus radiatus sprouts together with excipients suitable for oral administration.

Specifically, a nutraceutical or food composition comprising the fermentation product of L. brevis (DSM 33682) obtained in the presence of a plant extract of Phaseolus or Phaseolus radiatus, preferably Phaseolus radiatus sprouts in the form of capsules, granules in sachets or solutions, or sticks for oral administration.

The composition may include additional components such as, for example, prebiotics, vitamins, such as vitamin A, vitamin D, vitamin K3, vitamins of group B, amino acids, and oligo-elements as zinc, selenium, and iron.

The excipients of the compositions of the invention according to the chosen form are known to the person skilled in the art and can be selected for example from the group consisting of diluents, glidants, buffers, stabilizers, lubricants, disintegrants, sweeteners, anti-caking agents and preservatives.

The diluent suitable for the preparation of the composition of the invention is selected from the group consisting of cellulose, microcrystalline cellulose, calcium phosphate, starch, kaolin, calcium sulphate anhydrous or hydrate or dihydrate, calcium carbonate, lactose, sucrose, mannitol, polysaccharides, glucans, xyloglucan, starches, natural gums, malt, gelatine and mixtures thereof.

The buffering agent or pH corrector is selected from the group consisting of: potassium or sodium salts, sodium or potassium hydroxide and mixtures thereof.

The gliding agent may be selected from the group consisting of: talc, microcrystalline cellulose, and magnesium carbonate.

The lubricating agent suitable for the preparation of the composition of the invention is selected from the group consisting of glycerol dibenate, calcium or magnesium stearates, aluminium, sodium stearyl fumarate, hydrogenated vegetable oils, palmitic acid, alcohol, starch, mineral oils, polyethylene glycols, sodium lauryl sulphate, talc, glycerides, sodium benzoate and mixtures thereof.

The suitable disintegrating agent for the preparation of the composition of the invention is selected from the group consisting of cellulose derivatives such as sodium carboxymethyl cellulose, also known as carmellose, cross-linked carboxymethyl cellulose, also known as croscarmellose, hydroxypropyl methyl cellulose, hydroxypropyl cellulose phthalate, polyvinyl acetate phthalate, povidone, copovidone, and sodium starch glycolate.

The sweetening agent suitable for the preparation of the composition of the invention is selected from the group consisting of acesulfame potassium, maltodextrin, sorbitol, mannitol, isomalt, maltitol, lactitol, xylitol, aspartame, cyclamic acid, cyclamate salts, lactose, saccharin, and saccharin salts.

The anti-caking agent suitable for the preparation of the composition of the invention is chosen from the group consisting of: silicon dioxide and talc.

The preserving agent suitable for the preparation of the composition of the invention is chosen from the group consisting of: methylparabens, ethylparabens, sodium ethylenediaminetetraacetate, sodium benzoate, potassium sorbate and their mixtures.

The fermentation product obtained by the fermentation process can be in powder or granular form in an amount from 1 mg to 5 g in sachets.

The composition in orosoluble stick form includes the fermentation product obtained by the fermentation process according to the invention in an amount from 1 mg to 1 g together with an amount from 0.5 g to 1.5 g of a sweetening agent chosen among isomalt, aspartame, xylitol, lactitol, sodium cyclamate, dextrose, fructose, glucose, lactose and sucrose, 1 mg to 50 mg of a flavoring agent, 1 mg to 50 mg of an anti-caking agent chosen from colloidal silicon dioxide and talc, 0 mg to 50 mg of vitamins.

The composition obtained from the fermentation process can be in freeze-dried or granular form and stored in an amount from 1 mg to 1 g in capsules.

The composition in capsule form comprises the fermentation product obtained by the fermentation process according to the invention in an amount from 1 mg to 1 g, together with an amount from 0 to 20 mg of lubricating agent selected from one or more of talc, magnesium stearate, calcium stearate, stearic acid, hydrogenated vegetable oils, polyethylene glycol; 0 to 20 mg of anti-caking agent selected from silicon dioxide, talc; 0 to 300 mg of sweetening agent selected from sucrose, sorbitol, mannitol, saccharin, acesulfame, hesperidin, maltodextrin; 0 mg to 50 mg of vitamins.

It is an object of the invention a fermentation product as such or comprised in an oral composition for use in the treatment and/or prevention of intestinal inflammation.

The fermentation product or composition of the invention is useful in the treatment and/or prevention of inflammatory bowel diseases or disorders, for example, IBD, Crohn's disease, ulcerative colitis or diverticulitis.

The fermentation product or composition of the invention is useful for the treatment and/or prevention of ulcerative colitis. Preferably, the fermentation product or composition of the invention is for use in a subject affected by ulcerative colitis. More preferably, the fermentation product or composition of the invention is for use in a subject affected by mild to moderate ulcerative colitis.

The fermentation product or composition can be used as is in lyophilic form or added to food or pharmaceutically acceptable excipients to be formulated as capsules, soft capsules, tablets, orosoluble tablets, stick granules or sticks, or in solution.

The fermentation product or composition for use in the treatment and/or prevention of IBD, Crohn's disease, ulcerative colitis or diverticulitis according to the invention can be used alone or in combination or in association with concomitant therapies, in particular with anti-inflammatories. Preferably, the fermentation product according to the invention can be used alone or in combination or in association with Non-Steroidal Anti-Inflammatory Drugs (NSAIDs), preferably with mesalazine.

The composition of the invention can be administered for cyclic treatments and as maintenance therapy without any limitations.

The fermentation product or composition of the invention is administered 1 to 4 times daily, preferably 1 to 3 times daily, preferably 1 time daily.

The fermentation product or oral composition according to the invention can be administered in an amount from about 1×10⁸ CFU to about 1×10¹² CFU in humans one, two, three, four times daily for a treatment period of at least one week.

The fermentation product or composition according to the invention is for use in a subject affected by ulcerative colitis, or mild to moderate ulcerative colitis, unresponsive to mesalazine.

In the context of the present invention, the term treatment is meant to alleviate, reduce, ameliorate, or eliminate symptoms related to inflammatory bowel disorders.

### EXAMPLES

### Example 1: Determination of Anti-Inflammatory Activity of Plant Extracts In Vitro and In Vivo

Commercial plant extracts of the plants Scutellaria baicalensis Georgi, Boswellia serrata, Phaseolus radiatus sprouts, Curcuma longa, Camellia sinensis or broccoli sprouts (Botanical Cube, Inc.) were analyzed for their anti-inflammatory activity.
EX-1: Scutellaria baicalensis Georgi
EX-2: Phaseolus radiatus (sprouts)
EX-3: Curcuma longa
EX-4: Camellia sinensis
EX-5: Broccoli (sprouts)

Each plant extract was suspended in phosphate buffered saline (PBS) at a concentration of 1.2% (w/v), the pH adjusted to a value of 7.4 and the solution maintained at a temperature of 37° C. for 24 h under anaerobic conditions. After 24 hours, the solution showed no bacterial growth. Each solution was centrifuged for 20 min at 2500 rpm, the insoluble residues discarded and the supernatant filtered on 0.22-μm PES filters.

Table 1 reports the solution obtained for each extract.

TABLE 1

| Plant extract | Solution |
|---|---|
| Scutellaria baicalensis Georgi | Sol. Ex1 |
| Phaseolus radiatus (sprouts) | Sol. Ex2 |
| Curcuma longa | Sol. Ex3 |
| Camellia sinensis | Sol. Ex4 |
| Broccoli (sprouts) | Sol. Ex5 |

Evaluation of the anti-inflammatory activity of the plant extract solutions was carried out by in vitro and in vivo measurements.

### (a) Determination of the Efficacy of the Plant Extracts In Vitro by Measuring the Amount of IL-10.

IL-10 values were determined by ELISA assay according to Nemeth et al., J Immunol., 2005 175: 8260-8270. 3×10⁵ Cells/well of RAW264.7 macrophages (ATCC) were seeded on a 24-well plate (T=37° C., CO2=5% (v/v)). The cells were divided into 5 groups and each group was treated for 2 hours with 20 L of each of the Solutions obtained from the plant extracts (Sol. Ex1-Ex5).

Cells treated with mesalazine and the short-chain fatty acids (propionate, acetate and butyrate) were used as positive controls. 10 μl of lipopolysaccharide (LPS) from Sigma-Aldrich n.2630 (10 μg/mL) was added to each well and incubated at 37° C. in an incubator with CO₂=5% (v/v) for 24 hours to stimulate the immune response. At the end of incubation, the supernatants were collected and analyzed by ELISA assay (Invitrogen kit) to detect the amount of IL-10.

Table 2 shows the percentage values of IL-10 produced by RAW264.7 macrophages and the percentage increase compared with LPS.

TABLE 2

| | | % increase in |
|---|---|---|
| | % IL-10 (vs | comparison |
| Plant extract | LPS) ± SD | to LPS |
| Scutellaria baicalensis Georgi (Sol. Ex-1) | 48 ± 22 | 0.48 |
| Phaseolus radiatus (sprouts) (Sol. Ex-2) | 50 ± 22 | 0.5 |
| Curcuma longa (Sol. Ex-3) | 91 ± 27 | 0.91 |
| Camellia sinensis (Sol. Ex-4) | 0 | 0 |
| Broccoli (sprouts) (Sol. Ex-5) | 38 ± 12 | 0.38 |
| Mesalazine 0.1 mM | 216 ± 55 | 2.16 |
| Mesalazine 0.2 mM | 269 ± 18 | 2.69 |
| Acetate 5 mM | 199 ± 14 | 1.99 |
| Propionate 10 mM | 135 ± 2 | 1.35 |
| Butyrate 0.62 mM | 268 ± 71 | 2.68 |
| LPS | 100 | 0 |

### (b) Determination of the Efficacy of Plant Extracts In Vivo.

### Based on the Efficacy of the Plant in Example, c1

Ulcerative colitis was induced in a mouse model by administration of 2,4,6-trinitrobenzenesulfonic acid (TNBS) (Yang et al., Scientific Reports, 2016 6: 29716).

Male C57BL/6 mice aged 6-8 weeks were divided into groups of 8 mice each. Solutions obtained from the plant extracts (Sol. Ex-1-Ex-5) at a concentration of 1.2% (w/v) in PBS were administered 3 hours before induction with TNBS and then once daily for 3 days.
Group A: 100 μL ethanol 50% i.r.+PBS for gavage
Group B: TNBS 2 mg/mouse in 100 μL i.r.+PBS for gavage
Group C: TNBS 2 mg/mouse in 100 μL i.r.+Mesalazine 100 mg/kg gavage
Group D: TNBS 2 mg/mouse in 100 μL i.r.+Sol. Ex-1 (Scutellaria baicalensis Georgi)/600μL/mouse per gavage
Group E: TNBS 2 mg/mouse in 100 μL i.r.+Sol. Ex-2 (Phaseolus radiatus)/600 L/mouse per gavage
Group F: TNBS 2 mg/mouse in 100 μL i.r.+Sol. Ex-3 (Curcuma longa)/600 μL/mouse per gavage
Group G: TNBS 2 mg/mouse in 100 μL i.r.+Sol. Ex-5 (Broccoli sprouts)/600 μL/mouse per gavage.

Mice were sacrificed 6 h after the last administration by cervical dislocation. Table 3 shows the parameters of weight loss and colon length in mice at the end of treatment (day 3).

TABLE 3

| | | Body weight | Colon length |
|---|---|---|---|
| Animal group | Treatment | loss (%) ± SE | (mm) ± SE |
| Group A | Ethanolo + PBS | 100 ± 1.0 | 72 ± 2.1 |
| Group B | TNBS + PBS | 93 ± 0.7 | 55 ± 1.2 |
| Group C | TNBS + Mesalazine | 99 ± 0.4 | 68 ± 1.9 |
| Group D | TNBS + Sol. Ex-1 | 94 ± 1.0 | 58 ± 2 |
| | (Scutellaria b. Georgi) | | |
| Group E | TNBS + Sol. Ex-2 | 95 ± 1.0 | 64 ± 2 |
| | (Phaseolus radiatus) | | |
| Group F | TNBS + Sol. Ex-3 | 95 ± 0.9 | 58 ± 3 |
| | (Curcuma longa) | | |
| Group G | TNBS + Sol. Ex-5 | 95 ± 1.2 | 61 ± 3 |
| | (Broccoli sprouts) | | |

### Example 2: Characterization of Phaseolus Radiatus Sprout Matrix

Based on the efficacy of the plant from Example 1, Phaseolus radiatus extract (Ex-2), was selected among the plant extracts used and its antioxidant activity and reducing oxide potential were determined.

### (a) Determination of Antioxidant Activity.

Antioxidant activity was measured by the ORAC (Oxygen Radical Absorbance Capacity Assay) test (catalogue no. STA-345, Cell Biolabs kit), according to the supplier's instructions, by measuring the activity of the lipophilic portion, the hydrophilic portion and the total. Table 4 shows the antioxidant activity values obtained for the lipophilic portion, hydrophilic portion, and total.

TABLE 4

| | hydrophilic | lipophilic | Total |
|---|---|---|---|
| | ORAC (μmol | ORAC (μmol | ORAC (μmol |
| Plant extract | TE/g ± SD) | TE/g ± SD) | TE/g ± SD) |
| Phaseolus radiatus | 83.5 ± 1.4 | 40.9 ± 0.3 | 124.4 ± 1.7 |

### (b) Determination of Redox Potential

1.0 g of lyophilic extract of Phaseolus radiatus (Ex-2), was suspended in 100 mL of PBS, 10mM at pH 7.4 and kept under stirring for 30 min at 37° C.; the resulting suspension was centrifuged for 20 min at 9800 x rpm. The redox potential of the supernatant was measured using digital multimeter (HQ40D, Hach), equipped with a probe (Intellical™ MTC101, Hach). The redox potential value of Phaseolus radiatus sprout extract was 200.2 mV.

### Example 3: Fermentation of Lactobacillus brevis in the Presence of Phaseolus radiatus Sprout Extract

600 Liters of MRS medium (added with 12 g/l of Phaseolus radiatus (Ex-2) extract previously heat-treated (120° C. for 15 min in an autoclave) were inoculated with Lactobacillus brevis (DSM 33682), corresponding to a concentration of 1.5×10⁹ to 4.5×10⁹ CFU/ml. the solution was fermented for 12 hours at 37° C.+/−0.5° C. under mild agitation and anaerobic conditions. At the end of fermentation, the biomass was concentrated by centrifugation at 3000 rpm for 10 min at 4° C. to a concentration factor of 7+2 times. The obtained biomass was lyophilized.

A total of 6.7 kg of lyophilic product (Lio-Prob1) was obtained, having 3.5×10¹¹ to 8.0×10¹¹ CFU/g.

### Example 4: Fermentation of Lactobacillus brevis in the Absence of Plant Matrix (Comparison)

600 Liters of MRS medium were inoculated with Lactobacillus brevis (DSM 33682), corresponding to a concentration of 1.5×10⁹ to 4.5×10⁹ CFU/ml. the solution was fermented for 12 hours at 37° C.+/−0.5° C. under mild agitation and anaerobic conditions. At the end of fermentation, the biomass was concentrated by centrifugation at 3000 rpm for 10 min at 4° C. to a concentration factor of 7+2 times. The obtained biomass was lyophilized.

6.7 kg of lyophilic product having 3.5×10¹¹ to 8.0×10¹¹ CFU/g (Lio-Prob2) was obtained.

The Lio-Prob2 lyophilic was analysed in comparison with the product obtained by fermentation in the presence of Phaseolus radiatus (Example 3).

### Example 5: Determination of In Vitro Activity of L. brevis Fermentation Products in the Presence of Phaseolus Radiatus Sprout Extract

The anti-inflammatory activity of the fermentation product in lyophilic form of L. brevis DSM 33682 obtained in the presence of Phaseolus radiatus sprout extract (Lio-Prob1) according to Example 5, and the product obtained without Phaseolus radiatus sprout extract (Lio-Prob2) according to Example 6, were analyzed by means of determination of the amount of IL-1beta mRNA.

3×10⁵ Cells/well of RAW264.7 macrophages (ATCC) were seeded on a 24-well plate.

The cells were divided into three groups and incubated with 20 μl of the above lyophilic fermented products diluted in phosphate buffer to a final CFU/ml concentration of 6×10⁷ to 7.2×10⁸ or mesalazine (Sigma-Aldrich Y0000297) (0.1 mM). Ten μl of lipopolysaccharide (LPS) from Sigma-Aldrich n.2630 (10 μg/mL) was added to each well and incubated at 37° C. in a 5% CO₂ incubator for 24 hours.

At the end of incubation, macrophage RNA was extracted and analysed by Real-Time PCR to detect the gene transcription level of the proinflammatory cytokine IL-1beta, calculated as the mean value for the treated groups compared with macrophages treated with LPS alone. In particular, total RNA was isolated from each sample using the mini RNeasy kit (QiaGen GmbH, Hilden, Germany), and 1ug of total RNA was reverse transcribed by IScript™ cDNA Synthesis Kit (BioRad, Hercules, CA, USA). Real-time PCR was carried out by a BioRad CFX96 Touch™ Real-Time PCR Detection System using SsoAdvanced Universal SYBR Green super Mix (BioRad). The expression level of each mRNA was assessed using the ΔΔCT method, and Gapdh was used as housekeeping gene for normalization. The following primers were used.

IL-1beta fwd primer:
5′-CGAGGCAGTATCACTCATTG-3′;
IL-1beta rvs primer:
5′-CGTTGCTTGGTTCTCCTTGT-3′;
GAPDH fwd primer
5′-AACTTTGGCATTGTGGAAGG-3′;
GAPDH rvs primer
5′-CACATTGGGGGTAGGAACAC-3′.

Table 5 shows the level of inhibition of IL-1beta mRNA transcription by the tested products after analysis by Real-Time PCR.

TABLE 5

| | | | mRNA IL-1beta |
|---|---|---|---|
| | Phaseolus | | transcription |
| Bacterial | radiatus | | inhibition compared |
| strain | sprouts | Product | to LPS treatment |
| L. brevis | + | Fermentation Example 6 | 82% |
| DSM 33682 | | (Lio-Prob1) (6.8 × 10⁷ | |
| | | UFC/well) | |
| L. brevis | − | Fermentation Example 7 | 32% |
| DSM 33682 | | (Lio-Prob2) (7.2 × 10⁸ | |
| | | UFC/well) | |
| — | − | Mesalazine (0.1 mM) | 59% |

### Example 6: Determination of the In Vivo Efficacy of L. brevis Fermentation Product in the Presence of Phaseolus radiatus Sprout Extract

The efficacy of fermentation products of L. brevis DSM 33682 obtained in the presence of Phaseolus radiatus sprout extract (Lio-Prob1) or absence of the extract (Lio-Prob2) was determined by measurement of DAI score and colonic length.

Male C57BL/6 mice (Envigo) aged 6-8 weeks (8 mice/group), ulcerative colitis was induced with 2,4,6-trinitrobenzenesulfonic acid (TNBS) (Yang et al., Scientific Reports, 2016 6:29716).

Mice were divided into groups of 8 mice each and, starting 3 hours before TNBS administration and every 24 hours for the next three days, were treated orally with the freeze-dried fermentation product of L. brevis DSM 33682 obtained in the presence of Phaseolus radiatus extract (Lio-Prob1) obtained as in Example 3, or with the fermentation product of freeze-dried L. brevis DSM 33682 (Lio-Prob2) obtained as in Example 6.
Group A: 100 μL ethanol 50% i.r.+PBS per gavage
Group B: TNBS 2 mg/mouse in 100 μL i.r.+PBS per gavage
Group C: TNBS 2 mg/mouse in 100 μL i.r.+Mesalazine 100 mg/kg per gavage;
Group D: TNBS 2 mg/mouse in 100 μL i.r.+Lio-Prob1; 2 mg/500 μL/mouse per gavage;
Group E: TNBS 2 mg/mouse in 100 μL i.r.+Lio-Prob1; 1 mg/500 μL/mouse per gavage;
Group F: TNBS 2 mg/mouse in 100 μL i.r.+Lio-Prob1; 0.5 mg/500 μL/mouse per gavage;
Group G: TNBS 2 mg/mouse in 100 μL i.r.+Lio-Prob1; 0.25 mg/500 μL/mouse per gavage;
Group H: TNBS 2 mg/mouse in 100 μL i.r.+Lio-Prob1; 0.125 mg/500 μL/mouse per gavage;
Group L: TNBS 2 mg/mouse in 100 pL i.r.+Lio-Prob2; 0.8 mg/500 μL/mouse per gavage.

Mice were sacrificed by cervical dislocation 6 h after the last administration.

The disease activity index, expressed by DAI score, of C57BL/6 mice was measured according to the method reported by Yang et al., Scientific Reports, 2016 6:29716. The disease index is based on mean body weight loss, stool consistency, and the presence of bleeding in the stool (value 0=no effect on body weight, normal stool consistency, no bleeding in the stool; value 1=body weight loss between 1-5%; value 2=body weight loss between 5 and 10%, soft stools, slight bleeding; value 3=body weight loss between 10 and 15%; value 4=weight loss greater than 15%, diarrhea and bleeding).

Table 6 shows the DAI score and colon length for the treated animal groups.

TABLE 6

| | | | DAI | | | |
|---|---|---|---|---|---|---|
| | | UFC/ | score ± | | colon length | |
| Group | Treatment | mouse | SE | P value | (mm) ± SE | P value |
| Group A | Etanol + PBS | — | 0.1 ± 0.1 | | 70 ± 0.7 | — |
| Group B | TNBS + PBS | — | 3.8 ± 1.0 | | 49.8 ± 1.0 | — |
| Group C | TNBS + | — | 1.1 ± 0.5 | * | 66.6 ± 0.5 | **** |
| | Mesalazine | | | | | |
| Group D | TNBS + Lio-Prob1 | 7.0 × 10⁸ | 0.5 ± 0.4 | * | 69 ± 0.7 | **** |
| Group E | TNBS + Lio-Prob1 | 3.5 × 10⁸ | 0.9 ± 0.4 | * | 66.2 ± 0.4 | **** |
| Group F | TNBS + Lio-Prob1 | 1.7 × 10⁸ | 1.1 ± 0.5 | * | 63.4 ± 0.8 | **** |
| Group G | TNBS + Lio-Prob1 | 8.7 × 10⁷ | 1.3 ± 0.7 | * | 62.7 ± 0.5 | **** |
| Group H | TNBS + Lio-Prob1 | 4.3 × 10⁷ | 1.9 ± 0.6 | * | 56 ± 1.0 | * |
| Group L | TNBS + Lio-Prob2 | 5.7 × 10⁸ | 4.0 ± 1.4 | ns | 52 ± 0.4 | ns |

* P < 0.05,
**** P < 0.0001 vs Group B (ANOVA);
ns = not significant.

### Example 7: Dose Response Assay in Treated Mice

C57BL/6 Male mice aged 7-8 w (ENVIGO, Italy) were housed under standard laboratory conditions: light/dark cycles (12/12 hr), ambient temperature 20±2° C., 55% relative air humidity and food (Mucedola RF18) and water ad libitum.

After 7 days of adaptive feeding, mice were randomly assigned to different groups as follows: normal controls (Control group, n=8); untreated TNBS-induced colitis mice (TNBS group, n=8); TNBS-induced colitis mice subdivided according to the following groups of treatment, 8 mice for each treatment:
Group A: Ethanol solution+PBS
Group B: TNBS 2 mg/mouse+PBS
Group C: TNBS 2 mg/mouse+Mesalazine
Group D: TNBS 2 mg/mouse+Lio-Prob1 (80 mg/20 ml)
Group E: TNBS 2 mg/mouse+Lio-Prob1 (40 mg/20 ml)
Group F: TNBS 2 mg/mouse+Lio-Prob1 (20 mg/20 ml)
Group G: TNBS 2 mg/mouse+Lio-Prob1 (10 mg/20 ml)
Group H: TNBS 2 mg/mouse+Lio-Prob1 (5 mg/20 ml)

Colitis was induced in the mice as previously described, using TNBS (Yang et al., Scientific Reports, 2016 6:29716).

After 3 h of colitis induction, all animals were orally administered different treatments for 4 days. During the 4-day trial, the mice were routinely inspected for their body mass, and diarrhea. All mice were sacrificed by cervical dislocation for sample collection at the end of day 4.

The extent of gross macroscopic damage of colon was determined using previously established scoring system (Yang et al., Scientific Reports, 2016 6:29716; Morris et al., Gastroenterology 1989, 96, 795-803) (No damage: score 0; Hyperemia without ulcers: score 1; Hyperemia and wall thickening without ulcers: score 2; One ulceration site without wall thickening: score 3; Two or more ulceration sites: score 4; 0.5 cm extension of inflammation or major damage: score 5; 1 cm extension of inflammation or severe damage: score 6-10 (the score was increased by 1 for every 0.5 cm of damage up to a maximal score of 10).

After, colon tissues were collected and fixed into 4% buffered paraformaldehyde solution to be put in paraffine and stained with H&E staining solution according to known practice. Finally, the stained sections were observed and photographed under a light microscope. Colon mucosa damage index (CMDI) score was assessed in a blinded fashion (Siegmund et al. Am J Physiol Regul Integr Comp Physiol. 2001, 281, R1264-73; Obermeier et al., Clin Exp Immunol. 1999, 116, 238-245). CMDI was calculated by combining infiltration of inflammatory cells and the histological scores for tissue damage, ranging from 0 (no changes) to 6 (extensive cell infiltration and tissue damage) as follows:

| | Score |
|---|---|
| Infiltration of inflammatory cells | |
| Presence of occasional inflammatory cells in the lamina propria | 0 |
| Increased numbers of inflammatory cells in the lamina propria | 1 |
| Confluence of inflammatory cells, extending into the submucosa | 2 |
| Transmural extension of the infiltrate | 3 |
| Tissue damage | |
| No mucosal damage | 0 |
| Discrete lymphoepithelial lesions | 1 |
| Surface mucosal erosion or focal ulceration | 2 |
| Extensive mucosal damage and extension into deeper structures | 3 |
| of the bowel wall | |

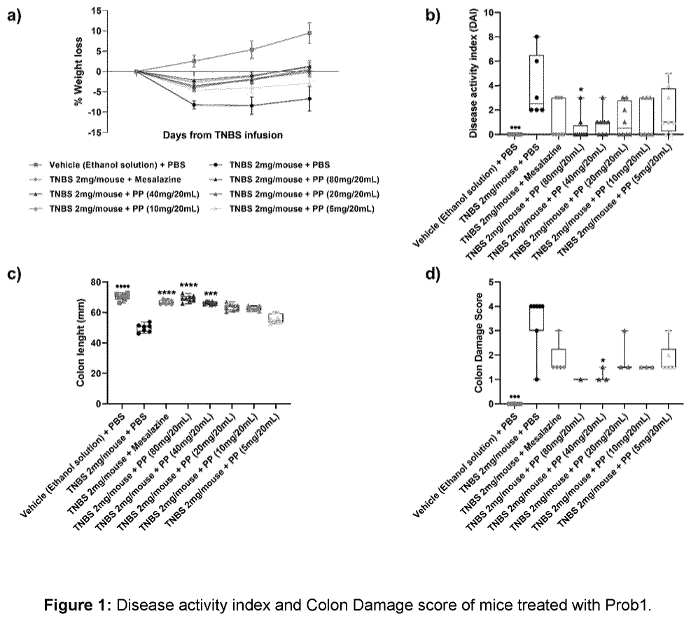

FIG. 1 shows the parameters of weight loss, DAI score, colon length and Colon Damage score for the treated groups of mice.

After an overall evaluation of the tests made with increasing doses of Lio-Prob1, according to Example 3, administered to mice model of ulcerative colitis, the dose of 12 mg/kg was selected being the dose having 50% of the improvement effect on the mice induced with ulcerative colitis.

A dose of 12 mg/kg in mice corresponds to a dose of 60 mg in humans.

### Example 8: Determination of Myeloperoxidase (MPO) Activity In Vivo on TNBS-Induced Colitis Mice

C57BL/6 Male mice aged 7-8 weeks (ENVIGO, Italy) were housed under standard laboratory conditions: light/dark cycles (12/12 hr), ambient temperature 20±2° C., 55% relative air humidity and food (Mucedola RF18) and water ad libitum.

After 7 days of adaptive feeding, mice were randomly assigned to different groups as follows: normal controls (Control group, n=8); untreated TNBS-induced colitis mice (TNBS group, n=8); TNBS-induced colitis mice treated with 100 mg/kg of mesalazine (n=8); TNBS-induced colitis mice treated with 12 mg/kg of Lio-Prob1 according to Example 3 (n=8).

Colitis was induced in the mice as previously described, using TNBS (Yang et al., Scientific Reports, 2016 6:29716).

After 3 h of colitis induction, each group of mice was orally administered with the corresponding treatment for 4 days. During the 4-day trial, the mice were routinely inspected for their body mass, and diarrhea. All mice were sacrificed by cervical dislocation for sample collection at the end of day 4.

Colonic mucosal scrapings from 1 cm of colon of mice were suspended in potassium phosphate buffer (pH 6.0) with hexadecyl trimethylammonium bromide buffer (Sigma Aldrich H5882) supplemented with a cocktail of protease inhibitors (Sigma Aldrich 539136). Samples were then homogenized on ice and sonicated. Then, the suspensions were centrifuged at 10000 g for 10 minutes at 4° C., and the supernatants were diluted in potassium phosphate buffer (pH 6.0) containing 0.167 mg O-dianisidine dihydrochloride (Sigma Aldrich D3252) and 0.0005% (vol/vol) H₂O₂. Changes in absorbance at 450 nm were recorded with a spectrophotometer (Tecan NanoQuant model Infinite M200) (Alex et al., Inflammatory bowel diseases 2009, 15, 341-352; Chin and Barrett, Dig Dis Sci. 1994, 39, 513-525).

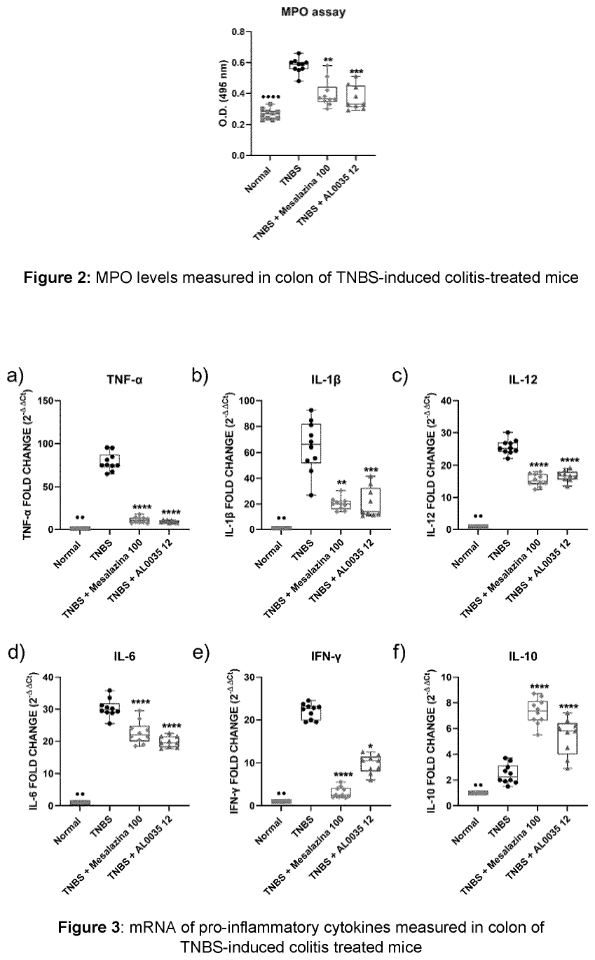

FIG. 2 shows the MPO values of the treated groups.

### Example 9: Relative Quantitative Real-Time PCR of Colonic Cells

RNA of colonic cells of C57BL/6 male mice treated according to Example 8 was isolated with Trizol (Life-technologies). First strand cDNAs were synthesized from 1 μg of total RNA in a 20 μl reaction with reverse transcriptase (Bi-oLine n. BIO 65053). Real-time PCR was performed using SYBR green Master Mix (#1725150, Biorad). GAPDH was used as internal control. The primers used are reported in Table 7. The relative transcription mRNA level was calculated.

TABLE 7
Primers list of qPCR experiments

| Gene name | Primer Sequence |
|---|---|
| FoxP3 | AGACCCCTGTGCTCCAAGTG |
| | CAGACTCCATTTGCCAGCAG |
| GATA3 | GAACCGCCCCTTATCAAG |
| | CAGGATGTCCCTGCTCTCCTT |
| MCP-1 | TGATCCCAATGAGTAGGCTGGAG |
| | ATGTCTGGACCCATTCCTTCTTG |
| NK1.1 | TCCCTTCTCACCACCAGTTA |
| | CAGTCTTGTGGGCACTCTAA |
| T-bet | AATCGACAACAACCCCTTTG |
| | AACTGTGTTCCCGAGGTGTC |
| IL-10 | ATTTGAATTCCCTGGGTGAGAAG |
| | CACAGGGGAGAAATCGATGAC |
| IL-6 | AGGATACCACTCCCAACAGACCT |
| | CAAGTGCATCATCGTTGTTCATAC |
| INF-γ | CAATGAACGCTACACACTGC |
| | CCACATCTATGCCACTTGAG |
| TNF-α | CCCCAAAGGGATGAGAAGTTC |
| | TGAGGGTCTGGGCCATAGAA |
| IL-1β | TCAGGCAGGCAGTATCACTC |
| | CTAATGGGAACGTCACACC |
| IL-12 | CAGAAGCTAACCATCTCCTGGTTTG |
| | TCCGGAGTAATTTGGTGCTTCACAC |
| GAPDH | AACTTTGGCATTGTGGAAGG |
| | CACATTGGGGGTAGGAACAC |

FIG. 3 shows mRNA of pro-inflammatory cytokines measured in colon of TNBS-induced colitis treated mice.

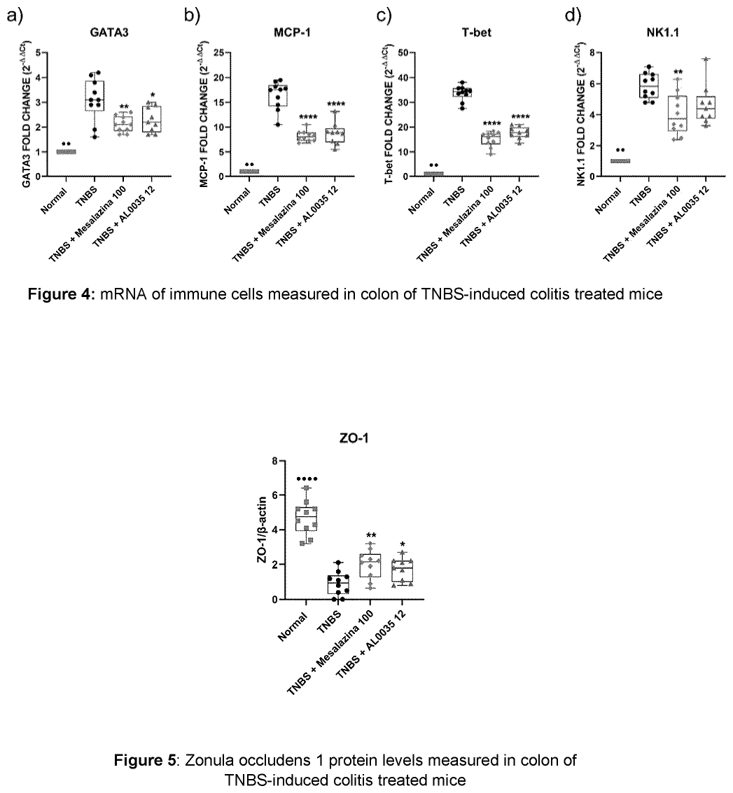

FIG. 4 shows mRNA of immune cells measured in colon of TNBS-induced colitis treated mice.

### Example 10: Western Blot Analysis of Colonic Cells

The mucosa of C57BL/6 male mice treated according to Example 8 was scraped from the colon. It was then immersed in urea extraction buffer (6 M Urea, 0.1% Triton X-100, 10 mM Tris, pH 8.0, 1 mM DTT, 5 mM MgCl2, 5 mM EGTA, 150 mM NaCl), supplemented with PMSF (Sigma Aldrich n. 93482) and an inhibitor cocktail of proteases (Cell Signaling n. 58715) to prevent protein degradation and sonicated for 25 seconds. Protein concentration was determined by Bradford assay according to the instruction of manufacturer (Biorad n. 5000006). Then, protein extraction samples were run on acrylamide gel under denaturing and reducing conditions. Different acrylamide concentrations were used according to the weight of the protein to be detected, as follows:
8% acrylamide gel for ZO-1 (Abclonal n. A0659) nd NK1.1 (Abclonal n. A8189)
10% acrylamide gel for Gata-3 (Abclonal n. A5711), T-bet (Abclonal n. A23414).
15% acrylamide gel for MCP1 (Abclonal n.A7277).

The proteins were then transferred to nitrocellulose filters, and unsaturated binding sites blocked with 5% non-fat milk for 1h. Filters were then incubated overnight at 4° C. with the antibody specific for the examined protein and with a species-specific HRP-conjugated secondary antibody (Invitrogen n. G21234). Actin and/or GAPDH were used as internal control. Immunoreactive bands were detected using a chemiluminescence kit according to the manufacturer's instructions (Life technologies), and the image acquired through a C280 Azure Biosystem documentation system. Densitometric analysis of the bands was performed by ImageJ software.

FIG. 5 shows ZO-1 protein levels measured in colon of TNBS-induced colitis treated mice.

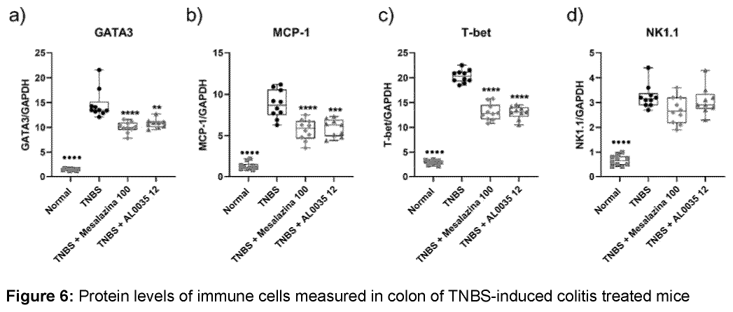

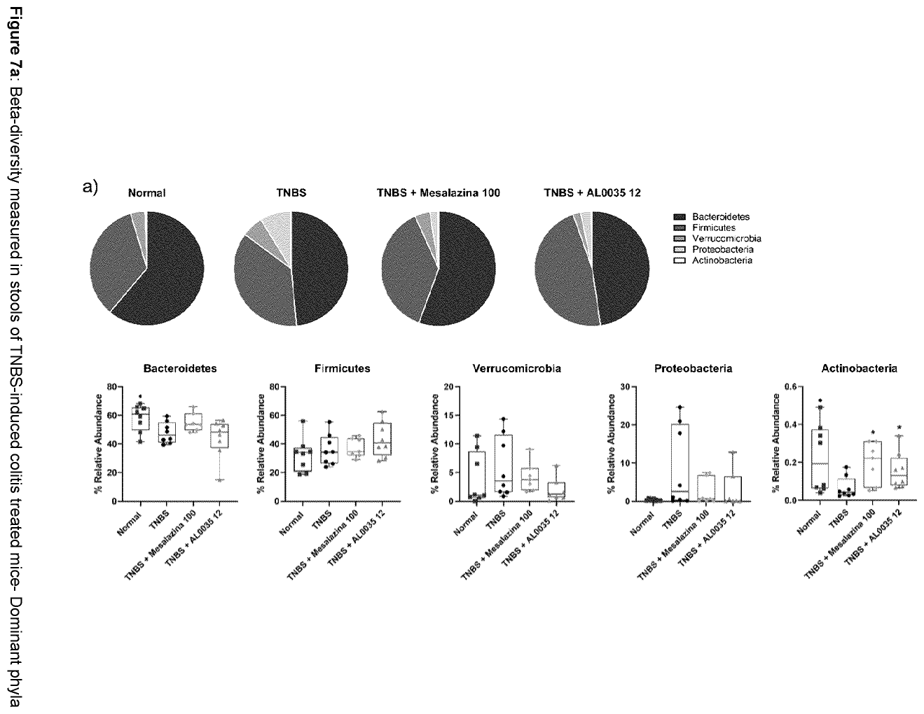

FIG. 6 shows the levels of immune cells and chemokine measured in colon of TNBS-induced colitis treated mice.

### Example 11: Fecal DNA Extraction and Luminal Microbiota Analysis

Feces of C57BL/6 male mice treated according to Example 8 were collected and stored at −80° C. after snap freezing in liquid nitrogen. The bacterial DNA of each sample was extracted using FastDNA SPIN Kit for soil and FastPrep Instrument (MP Biomedicals, Santa Ana, CA, USA) according to the manufacturer's protocols.

The V4-V5 hypervariable regions of the bacterial 16S rRNA gene were amplified and were sequenced for obtaining the microbial composition of the analyzed samples. Amplicon libraries were generated on with primers based the 515FB (5′-GTGYCAGCMGCCGCGGTAA-3′)/926R (5′-CCGYCAATTYMTTTRAGTTT-3′) (Walters, mSystems 2015, 1, e00009-15). The sequencing instrumentation, methodology, and chemistry were based on the Illumina MiSeq instrument using the 2×300 bp paired-end v3 chemistry as detailed by Comeau (Comeau et al., mSystems. 2017, 2: e00127-16).

All results were based on sequenced reads and operational taxonomic units.

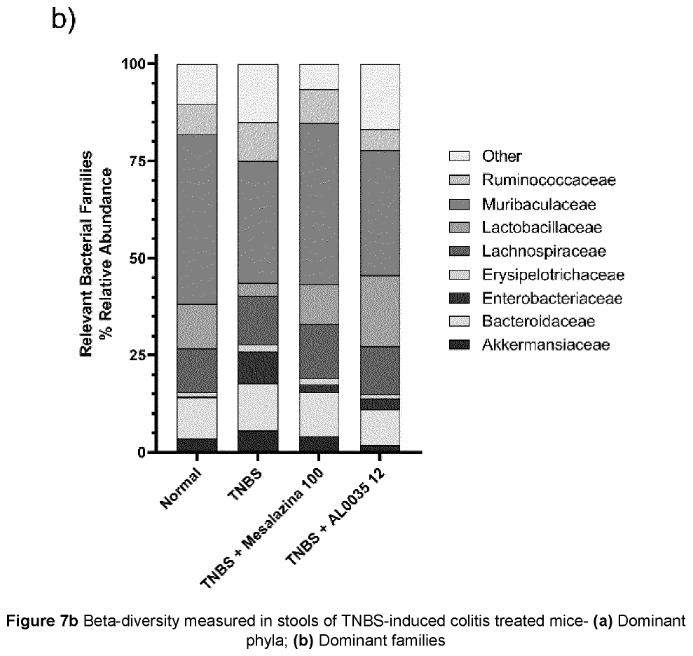

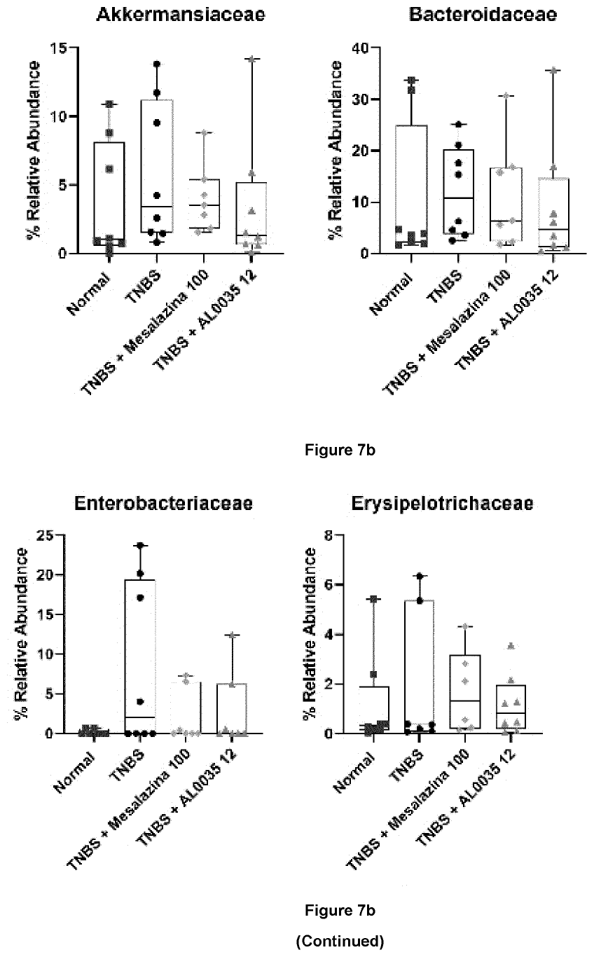

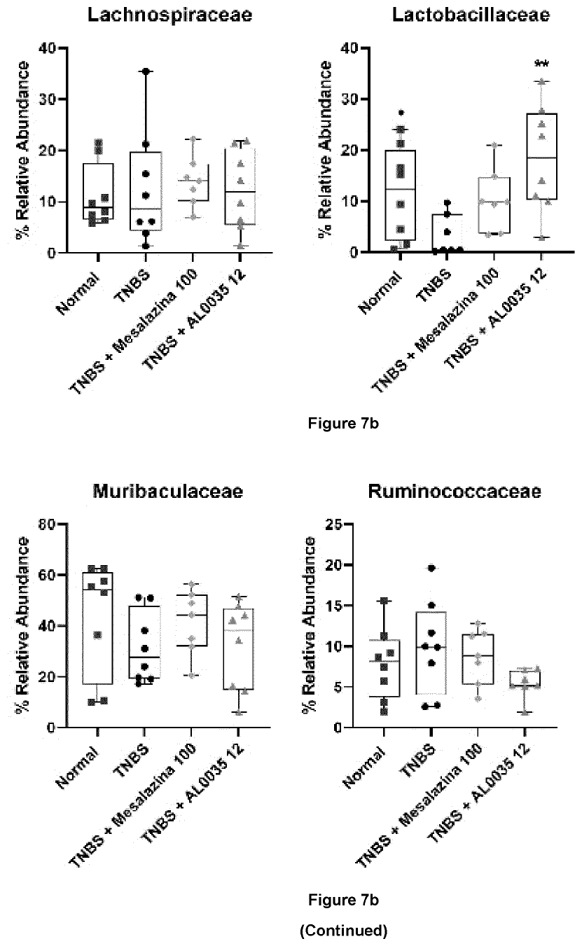

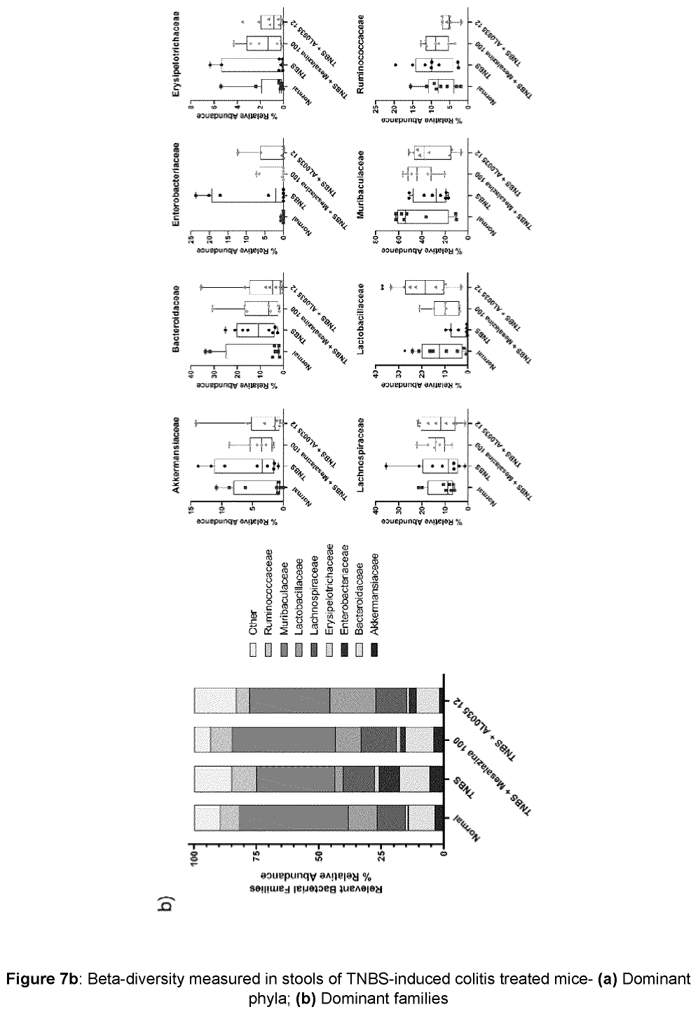

FIG. 7 shows the Beta-diversity analysis of intestinal microbioma measured in stools of TNBS-induced colitis treated mice

### Example 12: Determination of the In Vivo Efficacy of L. brevis Fermentation Product in the Presence OF Phaseolus radiatus Sprout Extract in Comparison to VSL #3 and E. coli Nissle

C57BL/6 Male mice aged 7-8 weeks (ENVIGO, Italy) were housed under standard laboratory conditions: light/dark cycles (12/12 hr), ambient temperature 20±2° C., 55% relative air humidity and food (Mucedola RF18) and water ad libitum.

After 7 days of adaptive feeding, mice were randomly assigned to different groups as follows:
Group 1: Vehicle (50% ethanol solution) ir+PBS 1 mL/mouse os
Group 2: TNBS 2 mg/mouse ir+PBS 1 mL/mouse os
Group 3: TNBS 2 mg/mouse ir+Mesalazine 1 mL/mouse (100 mg/Kg) os
Group 4: TNBS 2 mg/mouse ir+VSL #3 (Alfasigma) 5×10¹⁰ CFU/Kg/mouse os
Group 5: TNBS 2 mg/mouse ir+VSL #3 5×10⁹ CFU/Kg/mouse os
Group 6: TNBS 2 mg/mouse ir+VSL #3 5×10⁸ CFU/Kg/mouse os
Group 7: TNBS 2 mg/mouse ir+E. Coli Nissle (Cadigroup) 5×10¹⁰ CFU/Kg/mouse os
Group 8: TNBS 2 mg/mouse ir+E. Coli Nissle 5×10⁹ CFU/Kg/mouse os
Group 9: TNBS 2 mg/mouse ir+E. Coli Nissle 5×10⁸ CFU/Kg/mouse os os
Group 10: TNBS 2 mg/mouse ir+Lio-Prob1 5×10¹⁰ CFU/Kg/mouse os
Group 11: TNBS 2 mg/mouse ir+Lio-Prob1 5×10⁹ CFU/Kg/mouse os
Group 12: TNBS 2 mg/mouse ir+Lio-Prob1 5×10⁸ CFU/Kg/mouse os

Colitis was induced in the mice as previously described, using TNBS (Yang et al., Scientific Reports, 2016 6:29716).

After 3 h of colitis induction, each group of mice was orally administered with the corresponding treatment for 4 days. During the 4-day trial, the mice were routinely inspected for their body mass, and diarrhea. All mice were sacrificed by cervical dislocation for sample collection at the end of day 4.

Table 8 reports the body weight loss of the mice of different groups of treatment.

TABLE 8

| | | | | BWL |
|---|---|---|---|---|
| | | Dose mg or CFU | | body weight |
| Group | Product | per kg mouse | | loss % max |
| Group 1 | Negative control | 0 | | 0 |
| Group 2 | TNBS | 0 | | 12 |
| Group 3 | TNBS + mesalazine | 100 | mg | 4 |
| Group 4 | TNBS + VSL3 | 5 × 10¹⁰ | CFU | 8 |
| Group 5 | TNBS + VSL3 | 5 × 10⁹ | CFU | 9 |
| Group 6 | TNBS + VSL3 | 5 × 10⁸ | CFU | 12 |
| Group 7 | TNBS + Escherichia coli | 5 × 10¹⁰ | CFU | 7 |
| | Nissle | | | |
| Group 8 | TNBS + Escherichia coli | 5 × 10⁹ | CFU | 10 |
| | Nissle | | | |
| Group 9 | TNBS + Escherichia coli | 5 × 10⁸ | CFU | 8 |
| | Nissle | | | |
| Group 10 | TNBS + Lio-Prob1 | 5 × 10¹⁰ | CFU | 4 |
| Group 11 | TNBS + Lio-Prob1 | 5 × 10⁹ | CFU | 5 |
| Group 12 | TNBS + Lio-Prob1 | 5 × 10⁸ | CFU | 5 |

Table 9 reports the DAI score measured for each group of treatment, measured as reported in Example 6.

TABLE 9

| | | Mean of | | P |
|---|---|---|---|---|
| Group | Treatment | DAI score | S.E. | value |
| Group 1 | Vehicle (Ethanol solution) + PBS 1 mL(os) | 0.50 | 0.27 | — |
| Group 2 | TNBS 2 mg/mouse + PBS 1 mL (os) | 3.75 | 0.77 | — |
| Group 3 | TNBS 2 mg/mouse + Mesalazine 1 mL (100 mg/Kg) (os) | 1.43 | 0.61 | * |
| Group 4 | TNBS 2 mg/mouse + VSL#3 5 × 10¹⁰ (490 mg/20 mL/Kg) | 2.29 | 0.99 | ns |
| Group 5 | TNBS 2 mg/mouse + VSL#3 5 × 10⁹ (49 mg/20 mL/Kg) | 3.25 | 0.96 | ns |
| Group 6 | TNBS 2 mg/mouse + VSL#3 5 × 10⁸ (4.9 mg/20 mL/Kg) | 3.67 | 0.61 | ns |
| Group 7 | TNBS 2 mg/mouse + E. Coli Nissle 5 × 10¹⁰ (83.4 mg/20 mL/Kg) | 1.17 | 0.75 | * |
| Group 8 | TNBS 2 mg/mouse + E. Coli Nissle 5 × 10⁹ (8.34 mg/20 mL/Kg) | 3.29 | 0.89 | ns |
| Group 9 | TNBS 2 mg/mouse + E. Coli Nissle 5 × 10⁸ (0.83 mg/20 mL/Kg) | 2.63 | 0.91 | ns |
| Group 10 | TNBS 2 mg/mouse + Lio-Prob1 5 × 10¹⁰ (92 mg/20 mL/Kg) | 0.86 | 0.70 | * |
| Group 11 | TNBS 2 mg/mouse + Lio-Prob1 5 × 10⁹ (9.2 mg/20 mL/Kg) | 1.63 | 0.56 | * |
| Group 12 | TNBS 2 mg/mouse + Lio-Prob1 5 × 10⁸ (0.92 mg/20 mL/Kg) | 2.00 | 0.46 | * |

* P < 0.05 vs Group 2 (ANOVA);
ns = not significant.

Table 10 and reports the colon damage score measured for each group of treatment, measured as reported in Example 7.

TABLE 10

| | | Mean of | | |
|---|---|---|---|---|
| | | colon damage | | P |
| Group | Treatment | score | S.E. | value |
| Group 1 | Vehicle (Ethanol solution) + PBS 1 mL(os) | 0.05 | 0.00 | — |
| Group 2 | TNBS 2 mg/mouse + PBS 1 mL (os) | 3.63 | 0.18 | — |
| Group 3 | TNBS 2 mg/mouse + Mesalazine 1 mL (100 mg/Kg) (os) | 1.43 | 0.61 | ** |
| Group 4 | TNBS 2 mg/mouse + VSL#3 5 × 10¹⁰ (490 mg/20 mL/Kg) | 1.14 | 0.59 | *** |
| Group 5 | TNBS 2 mg/mouse + VSL#3 5 × 10⁹ (49 mg/20 mL/Kg) | 1.00 | 0.38 | *** |
| Group 6 | TNBS 2 mg/mouse + VSL#3 5 × 10⁸ (4.9 mg/20 mL/Kg) | 2.17 | 0.60 | * |
| Group 7 | TNBS 2 mg/mouse + E. Coli Nissle 5 × 10¹⁰ (83.4 mg/20 mL/Kg) | 0.67 | 0.49 | *** |
| Group 8 | TNBS 2 mg/mouse + E. Coli Nissle 5 × 10⁹(8.34 mg/20 mL/Kg) | 2.50 | 0.62 | ns |
| Group 9 | TNBS 2 mg/mouse + E. Coli Nissle 5 × 10⁸(0.83 mg/20 mL/Kg) | 1.00 | 0.42 | *** |
| Group 10 | TNBS 2 mg/mouse + Lio-Prob1 5 × 10¹⁰ (92 mg/20 mL/Kg) | 0.57 | 0.30 | *** |
| Group 11 | TNBS 2 mg/mouse + Lio-Prob1 5 × 10⁹ (9.2 mg/20 mL/Kg) | 1.00 | 0.50 | *** |
| Group 12 | TNBS 2 mg/mouse + Lio-Prob1 5 × 10⁸ (0.92 mg/20 mL/Kg) | 1.29 | 0.47 | *** |

* P < 0.05,
** P < 0.01,
*** P < 0.0001 vs Group 2 (ANOVA);
ns = not significant.

### Example 13: Characterization of the Supernatant of Fermentation of L. brevis DSM 33682 in the Presence of Phaseolus radiatus Sprouts by Mass Spectroscopy (LC-MS)

Each of samples Prob1 (fermented L. brevis DSM 33682 in the presence of Phaseolus radiatus sprouts), Prob2 (fermented L. brevis DSM 33682 in the absence of matrix) and Ex-2 (Phaseolus radiatus extract) was centrifuged (14000 rpm, 5 min at 4° C.) and diluted 1:10 with water.

10 μl of the sample were injected onto Acquity UPLC-BEH C18 column (1.7um; 100×2.1 mm, Waters).

Tests were performed on a liquid chromatography system coupled to a mass spectrometer (LC-MS) (Xevo G2-XS coupled to Acquity H-Class UPLC syste).

Chromatographic separation was achieved with linear gradient of water+0.1% (v/v) formic acid (Solvent A) and acetonitrile+0.1% (v/v) formic acid (Solvent B) at a flow rate of 0.3ml/min, as shown in Table 11.

TABLE 11

| Time (min) | % Solvent A | % Solvent B |
|---|---|---|
| 0 | 95 | 5 |
| 5 | 95 | 5 |
| 45 | 5 | 95 |
| 50 | 5 | 95 |
| 53 | 95 | 5 |
| 60 | 95 | 5 |

Ionization was carried out with an electrospray ionization (ESI) source. The following values were set:

| PARAMETER | VALUE | |
|---|---|---|
| Capillary | 0.80 | kV |
| Cone | 40 | V |
| Source temperature | 120° | C. |
| Desolvation temperature | 400° | C. |
| Gas flow in the cone | 25 | L/h |
| Desolvation gas flow | 500 | L/h |

Data acquisition was performed in MS scan in the ratio 50-2000 m/z in positive ion mode.

Qualitative analysis of the obtained chromatograms was performed, and a profile was created by subtraction of Prob2 and Ex-2 by Prob1 using Unifi 1.9.3.0 software (Waters), as shown in Table 12.

TABLE 12

| m/z (monoisotopic) | RT (min) | charge | MW (monoisotopic), Da |
|---|---|---|---|
| 575.384 | 39.06 | +1 | 574.376 |
| 605.395 | 44.27 | +2 | 1208.774 |
| 611.854 | 15.76 | +2 | 1221.692 |
| 633.344 | 15.96 | +2 | 1264.672 |

### Example 14: Analysis of Isoflavones Present in the Fermentation Product

A sample of the fermentation product of L. brevis DSM 33682 obtained in the presence of Phaseolus radiatus sprouts (Lio-Prob1) was solubilized in DMSO and the resulting solution was analyzed by HPLC according to the following conditions:
Column: C18 (Zorbax Eclipse XDB-C18 4.6×250 mm (5 μm))
Temperature: 35° C.
Flow rate: 1.0 ml/min
Injection volume: 5 μl
λ: 250 nm
Mobile phase A: water 0.1% HCOOH
Mobile phase B: acetonitrile

| Min | Phase A | Phase B |
|---|---|---|
| 0 | 90 | 10 |
| 20 | 70 | 30 |
| 25 | 50 | 50 |
| 30 | 25 | 75 |
| 31 | 90 | 10 |
| 35 | 90 | 10 |

Table 13 describes the percentage of isoflavones present in the sample.

TABLE 13

| Isoflavone | % |
|---|---|
| Daidzein | 0.0278 |
| Genistein | 0.00864 |
| Isoflavones(expressed as Daidzein) | 0.00436 |

### Example 15: Formulation in Sachet

The preparation of orosoluble sticks containing 60 mg or 240 mg of Lio-Prob1 fermentation product according to Example 3 is shown as an example. Table 14 shows the quali-quantitative composition.

TABLE 14

| COMPONENT | STICK 1 | STICK 2 |
|---|---|---|
| Lio-Prob1 fermentation product (Example 3) | 0.060 g | 0.240 g |
| Isomalt- E953 | 1.145 g | 0.965 g |
| Vanilla flavor | 0.017 g | 0.017 g |
| Silicon Dioxide (E551) | 0.015 g | 0.015 g |
| Cholecalciferol (vitamin D) (0.25%) | 0.013 g | 0.013 g |

### Example 16: Preparation of Capsule Compositions

Preparation of capsules containing 240 mg of Lio-Prob1 fermentation product according to Example 3 is shown as an example.

Table 15 shows the qualitative-quantitative composition.

TABLE 15

| | WEIGHT |
|---|---|
| COMPONENT | (mg) |
| Lio-Prob1 fermentation product (Example 3) | 240 |
| Maltodextrins | 91 |
| Cholecalciferol (vitamin D 0.25 percent) | 13 |
| Magnesium salts of fatty acids (magnesium stearate) E470b | 3 |
| Silicon dioxide (E551) | 3 |
| Vegetable capsule (hydroxypropyl methyl cellulose - E464 | 96 |
| and calcium carbonate - E170) | |

1. A fermentation product obtained by a fermentation process of the probiotic strain Lactobacillus brevis, deposited at the Leibniz Institute DSMZ-German Collection of Microorganisms and Cell Cultures GmbH, identified under the number DSM 33682, in the presence of at least one plant extract selected from the group consisting of: Scutellaria baicalensis Georgi, Boswellia serrata, Phaseolus, Phaseolus radiatus, Phaseolus radiatus sprouts, Curcuma longa, Camellia sinensis, broccoli sprouts and mixtures thereof.

2. The fermentation product according to claim 1 wherein the probiotic Lactobacillus brevis DSM 33682 is inoculated in an amount from 1×10⁸ to 1×10¹⁰ CFU/ml in a culture medium added with a plant extract selected from the group consisting of: Scutellaria baicalensis Georgi, Boswellia serrata, Phaseolus, Phaseolus radiatus, Phaseolus radiatus sprouts, Curcuma longa, Camellia sinensis, broccoli sprouts and mixtures thereof.

3. The fermentation product according to claim 2 wherein the plant extract is Phaseolus or Phaseolus radiatus or Phaseolus radiatus sprout extract at a concentration of 5 to 15 grams/litre than the fermentation solution.

4. The fermentation product according to claim 3 wherein the Phaseolus or Phaseolus radiatus or Phaseolus radiatus sprout extract is characterized by a reducing oxide potential of 100 to 400 mV when in phosphate buffer at concentrations from 0.5 to 20% (w/v).

5. The fermentation product according to claim 2 comprising compounds with molecular weights between 200 and 2000 Daltons.

6. The fermentation product according to claim 1 obtained by a process comprising the following steps:
(a) placing strain L. brevis DSM 33682 in culture medium in an amount of 1×10⁸ to 1×10¹⁰ CFU/ml;
(b) adding the Phaseolus extract or Phaseolus radiatus extract or Phaesolus radiatus sprouds extract to the culture medium at a concentration of 5 to 15 grams/litre;
(c) keeping under mild agitation in anaerobic or aerobic conditions at temperatures between 30° C. and 40° C. for 8 to 24 hours;
(d) centrifuging at speeds lower than 4000 rpm for a time from 5 to 40 minutes; and
(e) freeze-drying or freezing.

7. The fermentation product according to claim 1 comprising the fermentation product of Lactobacillus brevis DSM 33682 and the fermentation supernatant containing the bacterial metabolites of the fermentation carried-out in the presence of plants.

8. A nutraceutical or food composition comprising the fermentation product of L. brevis DSM 33682 obtained in the presence of a Phaseolus or Phaseolus radiatus or Phaseolus radiatus sprout extract according to claim 1 together with excipients suitable for oral administration.

9. The composition according to claim 8 further comprising prebiotics, vitamins, amino acids and mineral salts selected from salt of selenium, zinc and iron.

10. The composition according to claim 8 in the form of an orosoluble stick wherein the fermentation product of L. brevis DSM 33682, obtained in the presence of an extract of Phaseolus radiatus sprout is in an amount ranging from 1 mg to 1 g together with an amount from 0.5 g to 1.5 g of a sweetening agent selected from isomalt, aspartame, xylitol lactitol, sodium cyclamate, dextrose, fructose, glucose, lactose and sucrose, 1 mg to 50 mg of a flavouring, 1 mg to 50 mg of an anti-caking agent selected from colloidal silicon dioxide and talc, 0 mg to 50 mg of vitamins.

11. The composition according to claim 8 in form of capsule wherein the fermentation product of L. brevis DSM 33682 obtained in the presence of a Phaseolus radiatus sprout extract is in an amount from 1 mg to 1 g, together with an amount of 0 to 20 mg of lubricating agent selected from one or more of talc, magnesium stearate, calcium stearate, stearic acid, hydrogenated vegetable oils and polyethylene glycol; 0 to 20 mg anti-caking agent selected from silicon dioxide and talc; 0 to 300 mg sweetening agent selected from sucrose, sorbitol, mannitol, saccharin, acesulfame, neohesperidine and maltodextrin; 0 mg to 50 mg vitamins.

12. A method of treating and/or preventing intestinal inflammation in patients in need thereof with the composition according to claim 8, said method comprising
administering to said patients a pharmaceutically effective amount of said composition.

13. The method according to claim 12, wherein said intestinal inflammation comprises IBD, Crohn's disease, ulcerative colitis or diverticulitis.

14. The method according to claim 12, wherein the composition is administered alone or in combination or in association with concomitant therapies.

15. A process for obtaining the fermentation product of L. brevis DSM 33682 in the presence of Phaseolus or Phaseolus radiatus or Phaesolus radiatus sprout extract comprising the following steps:
(a) placing strain L. brevis DSM 33682 in culture medium in an amount of 1×10⁸ to 1×10¹⁰ CFU/ml;
(b) adding Phaesolus radiatus sprout extract to the culture medium at a concentration of 5 to 15 grams/litre;
(c) keeping under mild agitation in anaerobic or aerobic conditions at temperature between 30° C. and 40° C. for 8 to 24 hours;
(d) centrifuging at speeds below 4000 rpm for 20 to 40 minutes; and
(e) freeze-drying or freezing.

16. A process for obtaining pharmaceutical or nutraceutical compositions comprising the fermentation product according to claim 1 in the presence of at least one plant extract together with pharmaceutical or food excipients.
